(19) United States
(12) Patent Application Publication
(10) Pub. No.: US 2011/0098553 A1
(43) Pub. Date: Apr. 28, 2011

(54) AUTOMATIC REGISTRATION OF IMAGES FOR IMAGE GUIDED SURGERY

(76) Inventors: Steven Robbins, Winnipeg (CA); Gordon Scarth, Winnipeg (CA); Azi Ben-Lavi, Winnipeg (CA); Shawn Schaerer, Winnipeg (CA); Nishant Balakrishnan, Winnipeg (CA)

(21) Appl. No.: 12/913,155

(22) Filed: Oct. 27, 2010

Related U.S. Application Data
(60) Provisional application No. 61/255,735, filed on Oct. 28, 2009.

(51) Int. Cl. A61B 5/055

(52) U.S. Cl. 600/410

(57) ABSTRACT

Automatic registration of an MR image is carried out in an image guidance system by placing MR visible markers at known positions relative to markers visible in a camera tracking system. The markers are carried on a common fixture which is attached to a head clamp together with a reference marker which is used when the markers are covered or removed. The tracking system includes a camera with a detection array for detecting visible light a processor arranged to analyze the output from the array. Each object to be detected carries a single marker with a pattern of contrasted areas of light and dark intersecting at a specific single feature point thereon with an array around the specific location to allow the processor is able to detect an angle of rotation of the pattern and to distinguish each marker from the other markers.

This application claims the benefit of Priority under 35 USC 119 from Provisional Application 61/255,735 filed Oct. 28, 2009.

This invention relates to a method of registration of an image of a body part of the patient obtained in an imaging system to a position of the patient in a tracking system used in image guided surgery. The imaging system with which the techniques shown herein are to be used is preferably MRI but other systems including particularly CT imaging are also applicable.

This application is related to copending U.S. application Ser. No. 12/907,398 filed Oct. 19, 2010 which claims priority from Provisional Application 61/253,330 filed Oct. 20, 2009, the disclosures of which are incorporated herein by reference.

### BACKGROUND OF THE INVENTION

Physicians using an image guidance system during surgery need to relate locations of the patient's anatomy to the image data. This is done via a process called registration and usually, in a manual process, involves touching fiducials with a locating system wand and specifying what image pixel corresponds to the location of the fiducial. Specifying the location of a minimum of three fiducials is theoretically enough to allow a computer system to determine the appropriate co-ordinate transform between the image co-ordinates and the physical co-ordinates of the patient's anatomy. It is preferred to specify more than three fiducials to minimize the errors in the co-ordinate transform. This process is called manual registration because you must manually specify the pixel that corresponds to the fiducial and manually locate the fiducial.

It is preferred that the procedure is automated to eliminate the time required to perform the manual procedure. The manual procedure is typically performed after the patient has been anesthetised, and any reduction in time under general anaesthesia is preferred for the patient. Automating the procedure also reduces the possibility of error, which could lead to incorrect co-ordinate transformations. Incorrect co-ordinate transformations are typically detected by the image guidance system so the likelihood of an error leading to incorrect patient care is low, but the staff are then required to repeat the manual registration procedure a second time, which further adds to the time the patient is under general anaesthesia.

Further, procedures done with repeated intra-operative imaging sequences can require multiple registrations, so automating the procedure can provide benefits for each time registration is required.

An Image Guided Surgery System (IGS) provides a surgeon with spatial information. Typically, an IGS is used to indicate to the surgeon where the end or parts of a surgical tool is within or around the human body. For example, during brain surgery, one end of the tool might be in the surgeon's hand and the other end of the tool could be inside the patient, when it would not normally be visible. An IGS system finds the end of the tool that is outside the body, calculates (from tool geometry) where the other end of the tool is, and then registers the location of the tool with the prior images of the interior of the brain. This information is placed on a screen, allowing the surgeon to see what part of the brain the tool is affecting. This on-screen information, however, is composed of two merged datasets, that is a prior CT or MR image of the interior of the brain and the current position of the tool.

The prior art for auto registration comes from four main concepts:

1. Fusion with pre-operation images. This arrangement has the disadvantages that it is inherently inaccurate from the fusion process since the pre-op and intra-op images typically have differences in them (moved tissue, etc). Some manual intervention is usually required to correctly align the images. Also, any error from the first registration with the pre-op images is added to with the second registration, creating a larger overall error.

2. MR markers are installed on an anterior coil in the coil and infra-red markers are located outside. This arrangement has the disadvantages that the anterior coil can move after it has been placed around the patient due to a number of sources including imperfect fit, and this can create large registration errors. Since the frame is attached to the coil, it needs to be a relatively small size which gives larger errors on average than a larger frame.

3. MR and infra-red markers are mounted on a frame that is strapped to the patient just before imaging, and removed just after imaging. This is the same as the above with the only change being that the frame is not attached to the coil.

4. Fiducials on the face of the magnet. Because the long distance to the tracking volume, small errors in the localization of the magnet markers are amplified to large errors in the tracking volume

The following patents relate to this field:

U.S. Pat. Nos. 5,662,111 issued Sep. 2, 1997; 6,006,126 issued Dec. 21, 1999; 6,351,661 issued Feb. 26, 2002 and 6,405,072 issued Jun. 11, 2002 all by Cosman and issued to Radionics disclose a system for quantitative computer graphic determination of positions on a patient's anatomy and positions on associated equipment located near the patient's anatomy in relation to anatomical data, as from CT or MR scanning. A first camera produces a quantitative electronic readout of its field-of-view which provides a determination of relative spatial co-ordinates of uniquely identifiable points in its field-of-view. A second camera produces a quantitative electronic readout of its field-of-view which provides a determination of relative spatial co-ordinates of uniquely identifiable points in its field-of-view. The two cameras are located with respect to the patient's anatomy and the associated equipment so that the fields-of-view of the cameras include both the patient's anatomy and the equipment, but are taken from different directions. A body marker is positioned with respect to the patient's anatomy at a known position relative to said patient anatomy. The body marker has known co-ordinates in a stereotactic co-ordinate system that is established relative to the patient's anatomy so that the co-ordinates of all identifiable points in the fields of view of the two cameras can be determined relative to the stereotactic co-ordinate system and related to imaging data.

U.S. Pat. No. 6,026,315 (Lenz) issued Feb. 15, 2000 to Siemens relates to an apparatus for calibrating a navigation system in relation to image data of a magnetic resonance apparatus, positions of at least three markers arranged in an imaging volume of a magnetic resonance apparatus are determined with the navigation system in a first co-ordinate system, and are determined by means of magnetic resonance in a second co-ordinate system. From the positions of the markers in the two co-ordinate systems, a position and an orientation of the two co-ordinate systems to one another are determined. Localization data are transformed into the second co-ordinate system. An apparatus for conducting the method has at least one marker having a substance that can be detected using magnetic resonance technology, in spatial allocation to optical markings. A pickup coil can be spatially allocated to each marker.

U.S. Pat. No. 6,609,022 (Vilsmeier) issued Aug. 19^{th} 2003 to BrainLAB relates to intra-operative image updates for image guided surgery in which the technique of image fusion is used on a second data set relative to an original or first data set as a means of updating the image guidance data set for the navigation system. Registration is done using a reference frame that can be detected by both the tracking system and the imaging system.

U.S. Pat. No. 6,714,629 (Vilsmeier) issued Mar. 30^{th} 2004 to BrainLAB relates to registration between a tomographic data set and X-ray images acquired intra-operatively.

U.S. Pat. No. 6,584,174 (Schubert) issued Jun. 24^{th} 2003 to BrainLAB relates to registering information from an imaging system into a navigation system in which the position of the imaging system is detected by the navigation system for automatic registration updates by comparing a new image with a previous image to determine any changes and re-registering in the event changes are detected.

U.S. Pat. No. 6,490,473 (Katznelson) issued Dec. 3^{rd} 2002 by Coin Medical Technologies uses fiducials positioned at fixed points relative to the imaging system and to a wand, which can then be detected by the tracking system, so that the image co-ordinate system is registered to the tracking system. The system thus provides reference points positioned in predetermined location relative to the co-ordinate set of the scanning apparatus.

U.S. Pat. No. 6,516,213 (Neva) issued Feb. 4^{th} 2003 to Robin Medical uses small sensor coils inside the magnetic field of an MRI magnet to detect the location and orientation of the sensor coils using the magnetic field gradients generated during MR Imaging.

U.S. Pat. No. 6,871,086 (Neva) issued Mar. 22^{nd} 2005 to Robin Medical as a Continuation-in-Part of the above patent uses the same small sensor coils inside the magnetic field of the MRI magnet to detect the location and orientation of an endoscope using the magnetic field gradients generated during MR Imaging.

U.S. Pat. No. 6,381,485 (Hunter) issued Apr. 30, 2002 to Surgical Navigation (a division of Medtronic) discloses an auto-registration system in which fiducial markers are placed on a patient during imaging so that the position in the image of the fiducial markers is known. The system then uses electromagnetic sensors which are placed on the patient in a predetermined location relative to the fiducial markers. The electromagnetic sensors are then located in a magnetic field generated around the patient to locate the sensors in an image data set. As the position of the fiducial markers is known relative to the sensors, the images can then be registered.

There are many techniques for tracking the tool in the tracking system. Typically these use an array of infra-red reflective elements mounted on the tool which are visible in a detected infra-red image and the position and orientation of the tool can be determined from the analysis of the location of the elements in the image.

Another arrangement which uses a conventional camera system using visible light is shown in U.S. Pat. No. 6,978,167 (Dekel) issued Dec. 20, 2005 to Claron Technology Inc discloses a method for detecting and tracking the pose of an object such as a surgical tool displaceable in a co-ordinate reference frame. A visible target pattern on a marker includes a series of contrast regions of dark and light for providing feature points at which the dark and light regions meet at a juncture of an optically detectable plurality of edges. The method and system determine the location of the feature points by first locating the edges using the change in contrast between contrast regions, and then determining junctures of multiple edges. A stereoscopic digital camera generates a pair of digital images of the target pattern and a marker template comprising a set of reference characteristics including a relationship between the feature points.

This patent discloses in detail a method for detecting the feature point at the junction of the contrasting regions, which method is particularly applicable herein so that the details of this patent are hereby incorporated by reference or may be referred to for further detail of the method.

U.S. Pat. No. 5,828,770 (Leis) issued Oct. 27, 1998 to Northern Digital Inc discloses a system for determining the spatial position and angular orientation of an object in real-time is provided having a sensor section and a plurality of markers which emit a detectable energy. The markers are activated in an initial marker-identification mode. With such system, because the markers have been each uniquely identified during the marker-identification mode, and the relative marker geometry is known, the markers are simultaneously activated, detected and tracked during a subsequent marker-tracking mode.

U.S. Pat. No. 5,923,417 (Leis) issued Jul. 13, 1999 to Northern Digital Inc. discloses a similar system which includes a common energy detector for detecting both the energy emitted by an active target and the energy reflected by a passive target.

Existing state-of-the-art tracking technologies claim to have an accuracy in the 2 mm range. These devices typically use infra-red or other non-visible optical tracking technology and suffer from a number of limitations including reduced capabilities with angulations of the tracked tool, difficulties in positioning a reference frame for tracking, inaccuracies from the positioning of the tracking reference as compared with the tracking field of view, and inaccuracies causes be contaminants (e.g., fingerprint on IR tracking sphere). These common problems lead to poor utility and inaccuracies in tracking tools during surgery.

Relying on multiple markers does decrease the system error (smaller inaccuracy), but increases the footprint/size of the tracked device and can lead to poor ergonomic design of tracked devices.

The standard markers that are available on the market today (IR spheres, X Points) do not have sufficient information associated with each marker to allow the use of a single marker (one IR sphere or X Point) to be used for tracking an object/device. This information only allows for a tracking system to measure the translational information of a single marker, since the system cannot determine rotational information from a sphere or symmetrical pattern. Tracking systems also have a problem distinguishing one marker from another, so the inclusion of multiple generic markers in a tracking systems field of view creates the problem of uniquely determining the identity of each marker and the object associated with it. If the objects are at rest the system could determine which object is associated with each marker, but if the objects are moving then the system will have a difficult time distinguishing one marker from another.

In order for these markers to be used in a tracking system multiple markers must be used and configured in a unique geometric pattern. Each unique pattern and associated markers are affixed to an object that needs to be tracked. This allows the tracking system to identify and track objects using multiple markers.

### SUMMARY OF THE INVENTION

According to a first aspect of the invention there is provided a method for registering images, comprising:

obtaining at least one image of a part of the body of the patient in an imaging system;

the imaging system defining an image co-ordinate system;

placing image visible positioning markers at positions on or adjacent the part of the patient so as to be visible in the image;

obtaining a series of location images of one or more elements adjacent part of the patient in an image guidance system for providing location information relating to the elements;

the image guidance system defining a location co-ordinate system;

placing at least one guidance system positioning marker visible in the location image at respective positions on or adjacent the part of the patient so as to be visible in the location image, the image visible positioning markers being located at predetermined known locations relative to said at least one guidance system positioning marker in the image guidance system;

and providing automatic registration between the image co-ordinate system and the location image co-ordinate system by using data relating to the relative positions of the image visible positioning markers and said at least one guidance system positioning marker.

The arrangement defined above and described in more detail hereinafter is primarily designed for use in MR imaging but can be used also in other types of imaging including but not limited to X-ray, CT, PET.

In one preferred arrangement, the image visible positioning markers and said at least one guidance system positioning marker are separate markers independent of one another and are placed on a common registration element.

The common registration element, commonly described hereinafter as a fiducial frame sheet, and which in some embodiments is defined by a series of flat panels, is not visible in the MR imaging so that the markers themselves can be viewed in the MR image.

Also, if the common registration element or fiducial frame is radiolucent so that it does not show up in X-ray images, then it can also be used in a system which uses a combination of X-ray (or CT) and MR imaging to obtain images for use in surgical or interventional procedures. Such a system is described in more detail in PCT application PCT/CA2009/000672 filed May 25, 2009 which corresponds to U.S. application Ser. No. 12/420,859 filed Apr. 9, 2009 and published Dec. 10, 2009 as 2009/0306494, the disclosure of which is incorporated herein by reference.

Preferably the common registration element is located underneath the part of the patient such that the element can remain in place during the imaging and the location imaging.

Preferably the image is obtained by a magnetic resonance imaging system including a magnet, an RF transmission and receiving system and an image generating and control system for operating the magnet and the RF system to generate an MR image of the body part of the patient.

Preferably the part is the head and the common registration element forms a sheet which is located beneath the head of the patient. However the common fixture can be located at different places relative to the patient.

Preferably the common registration element forms a curved sheet with an upwardly facing concave surface with an axis of curvature extending longitudinally of the patient.

Preferably the image visible positioning markers are located in the sheet.

Preferably said at least one guidance system positioning marker is located at an end face of the sheet.

Preferably the common registration element markers are located on the common registration element so as to face upwards on either side of the patient.

Preferably the sheet is formed of a plastics material which is not visible in the image with a pattern of drilled holes containing an image visible material.

Preferably the common registration element is attached to a head fixation device.

Preferably the head fixation device is arranged for attachment to an end of a patient table.

Preferably the common registration element is arranged such that it can be moved into place after the patient is pinned into the head fixation device and can remain for the duration of the operation. Thus the common registration element is used or can be used in both a pre-operation scan and/or in intra-operative scans.

Preferably the imaging system is an MR system including a posterior RF coil and wherein the common registration element is separate from the posterior coil.

Preferably the posterior coil is arranged to fit underneath the fixture and to be removable while the common registration element remains in place.

Preferably the imaging system is an MR system including a posterior RF coil and wherein the common registration element is arranged to clamp onto the posterior coil.

Preferably the image guidance system comprises a visible optical system using cameras for detecting the positioning markers using visible light.

Preferably the image visible positioning markers are arranged in a non-regular array.

Preferably the non-regular array is arranged to allow location of the array relative to the common registration element when only part of the common fixture is visible in the MR image.

Preferably the non-regular array is different for different ones of a plurality of common registration elements and a determination is made as to the different fixtures by analyzing the array.

Preferably the common registration element has an opening through the common fixture defining an access port for accessing the body part of the patient.

Preferably there is provided a separate fixed reference having a visible positioning marker, the location of which can be determined in the image guidance system.

Preferably the separate fixed reference is used for the image guidance system rather than the markers after the location of the reference relative to the markers has been determined by the image guidance system.

Preferably the separate fixed reference remains exposed when the patient is draped for surgery.

Preferably there is provided a plurality of divots or recesses that can be located by physical contact through the drapes.

Preferably the separate fixed reference includes one or more markers on the skull clamp.

Preferably the elements detected in the series of images are tools for use by a surgeon where the series of images provides guidance information to the surgeon.

Preferably the MR imaging is carried out intra-operatively to obtain a new MR image and registration of the new MR image with the guidance image is carried out automatically, that is without manual intervention, for each MR image set acquired

Preferably the imaging system is an MR system including an RF coil, wherein the RF coil is arranged to flex and wherein the common registration element includes a plurality of flat panels attached to the flex coil.

Preferably the RF coil is arranged to flex in one direction only and the flat panels form a plurality of side by side strips.

Preferably the RF coil is arranged to flex in two directions only and the flat panels form an array of flat panels in rows and columns.

Preferably the flat panels each include at least one guidance tracking marker and at least one image tracking system marker.

Preferably the system is arranged to detect and correct for movement of one of flat panels using the tracking system markers on each flat panel.

Preferably the common registration element includes a plurality of fiducial frames, for example one on the top coil and one on the bottom coil.

Preferably the system is arranged to detect and correct for movement of one of the fiducial frames using the tracking system markers on each fiducial frame.

Preferably the image visible positioning markers are located in an image of the part of the patient by carrying out at least one separate marker-sensing scan used for locating only which scan is separate from one or more anatomical scans which are used to generate an anatomical image of the part of the patient.

Preferably the image is obtained by MR imaging and wherein the separate marker sensing scan is carried out using separate MR coils for the scan.

In another preferred arrangement, the system uses a number of attachable fiducial markers arranged to be attached to the patient that can each be detected in the imaging system and by the tracking system.

Preferably this system uses marker recognition to perform automatic registration.

Preferably there is provided in this system a spacer not visible in imaging to ensure that the image marker is located away from the skin during imaging.

Preferably in this system the algorithm is arranged with an arbitrary arrangement of such attachable markers.

Preferably in this system the head is not pinned during imaging, but the attachable fiducial markers are kept in place between the time of the scan and the time at which the patient is on the table and pinned.

The attachable markers can be attached using adhesive, screw into bone, or the like.

In one preferred arrangement, the markers are carried on at least one plate carried on a mounting device which is moved to provide adjustment of the position of the plate relative to patient while holding the plate stationary during the imaging.

In this arrangement, the plate and the mounting device are removed after imaging.

In this arrangement, there is a pair of plates each mounted on a respective side of the patient.

In this arrangement, the mounting device is removably mounted on a head clamp.

Alternatively the mounting device can be mounted on a robotic arm which allows it to be moved by the movement of the arm under control of the operating system from a deployed position to a retracted position.

There are two kinds of automatic registration described herein. The first utilizes a fiducial frame. This kind of auto registration can be applied to either pre- or intra-operative scans. The key feature is that the patient's head must be pinned prior to the scan.

The second kind of automatic registration uses a number of attachable fiducial markers (using adhesive, screw into bone, or the like) that can each be detected in the MR/CT and by the tracking system. Using marker recognition, the system can perform automatic registration. Additionally, the system may opt to include a spacer not visible in imaging to ensure that the MR/CT marker is located away from the skin during imaging; this helps make the automatic marker recognition more robust. The algorithm for this implementation of automatic registration works with an arbitrary arrangement of such markers. This kind of auto registration does not require the head to be pinned, but does require that the fiducial markers are kept in place between the time of the scan and the time at which the patient is on the table and pinned.

While optical tracking technology is described in detail herein, other forms of tracking can be used as are known to persons skilled in this art. For example an alternative arrangement which can be used is that of electromagnetic tracking. In this system, the marker is defined by a small coil which can be as small as ⅛ inch or smaller. The system then provides a source of EM radiation at a location in the room to generate an EM field and detects the effect of the field at the coils.

In MR imaging, MR markers of any kind can be used to replace the markers of MR visible material described herein including: a passive signal source (e.g. protons), a small coil that senses some feature of the MR (e.g. magnetic gradient fields), and a small coil that produces an MR signal when energized.

When using MR as the imaging system, the markers visible in the MR image can be scanned to determine their location in the image in a separate marker-sensing scans which is separate from the subsequent anatomical scan carried out to determine the location of the material in the body of the patient which is under scrutiny. The anatomical scans can be carried out in the same imaging session or at a different time. The MR imaging software can then put together the data from the images. For example, the system might use one pulse sequence to image the relevant anatomy at high resolution and a different pulse sequence to image the space outside the head where the markers are located. The system may even utilize different RF coils for the scans. The MR imaging system can control or drive the scanning in the separate marker sensing scan or the navigation system may be programmed to drive the scanner acquisition in this mode.

The system may use multiple fiducial frames, for example one attached on the top coil and one on the bottom coil. The system may use multiple fiducial frame segments on a single, flexible coil. Each fiducial frame segment in this arrangement includes a number of both MR and tracking system markers. In the limit, the system could use only a single marker that is visible to both the tracking system and to the MR imaging system.

The system can detect and correct for movement of one of the fiducial frames/fiducial frame segments using the tracking system markers on each fiducial frame/segment. The system can use the co-ordinate reference frame or add tracking system markers on the skull clamp to achieve the same purpose.

The arrangement as described in detail hereinafter may include one or more of the following features:
The ability to support automatic registration without the need to remove the auto registration frame;
Lower average error since fiducial frame rigidly attached to a rigid structure (HFD) minimizing movement (compared to on an MR coil that can introduce movement);
MR encoding markers and/or tracking system markers can be used to automatically identify the type of fiducial frame;
Placement of MR markers in patterns allows identification and localization of the markers when only a subset of markers are captured in the MR field of view;
The use of optical tracking for automatic registration with optical frame reference;
Enhanced workflow by automating the image registration;
Fiducial frame can be installed immediately after patient pinned and remain for the duration of the operation (it does not need to be removed);
Effectiveness of the fiducial frame not affected by draping;
Since the fiducial frame is independent of any MR coil, it is not dependent on coil placement or coil movement;
Proximity to the tracking volume allows lower average tracking error (i.e., error amplification minimized with a small distance to the tracking tool as compared to, for example, having markers out side of the bore);
An access port in the fiducial frame allows access to the patient
Ability to adjust the optical reference for optimal placement independent from the fiducial frame optical reference
MR encoding markers can be used to automatically identify the type of fiducial frame
Placement of MR markers in patterns allows identification and localization of the markers when only a subset of markers are captured in the MR field of view
The ability to automatically produce the co-ordinate mapping from a new set of MR images taken intra-operatively so that the surgeon can begin tracking immediately.

The embodiment described hereinafter provides an automatic registration system that can be used for both pre-operative and intra-operative MR images to provide the mapping between the optical tracking space and the MR imaging space without manual intervention for each MR image set acquired.

For the intra-operative case, this is accomplished with the introduction of a “fiducial frame” that lies within the MR coils but under the patient from the beginning to the end of the procedure, and is designed to be rigidly affixed to the HFD and not interfere with the patient.

The fiducial frame has special MR markers embedded in special shapes and geometries designed to provide high registration accuracy.

These markers may be designed such that if only a subset of the MR markers can be identified in the MR field of view, their location on the frame can be uniquely identified.

The MR markers may also be used to encode the type of fiducial frame used, allowing automatic identification of more than one frame with its associated geometry (e.g., for different sized fiducial frames for different sized patients). A fixed member of the fiducial frame contains optical markers that can be viewed by an optical camera at least at the beginning of the procedure (before patient draping is applied which could cover these markers). This fixed member with optical markers may be the same as the optical reference frame used for tracking or it may be independent. In either case, the optical markers on the fixed member may contain an encoding to uniquely identify it as the fiducial frame reference.

An optional access port can be included with the fiducial frame to allow access to the patient (e.g., for prone positioning).

As an alternative, for pre-operative MR images, optical markers visible in the camera system of the image guidance system are located on or adjacent MR visible markers that are placed in the MR field of view on the patient before pre-operative MR images are acquired. The optical markers at the MR markers allow the automatic identification of the MR markers in the optical co-ordinate space.

For both intra-operative and pre-operative MR images, a computer algorithm is used as part of the automatic registration system to automatically identify the optical and MR markers, and calculate the co-ordinate mapping between the two. This allows the automatic registration and therefore enables navigation without manual registration.

### BRIEF DESCRIPTION OF THE DRAWINGS

One embodiment of the invention will now be described in conjunction with the accompanying drawings in which:

FIG. 1 is a schematic illustration of a system according to the present invention showing a patient table, an MRI imaging system for generating an MR image and a tracking system for generating tracking images.

FIG. 2 is a schematic illustration of the tracking system of FIG. 1.

FIG. 3 is an isometric view of a patient table including a first embodiment of common fixture for the MR visible markers and the positioning marker or markers.

FIG. 4 is an isometric view of a second embodiment of common fixture which includes portions attached to an anterior coil and a posterior coil which are both of a flexible construction.

FIGS. 5, 6 and 7 show a front face of three embodiments of marker as used in the system of FIG. 1.

FIG. 8 shows an alternative arrangement for registration which includes markers located on the body of the patient.

In the drawing like characters of reference indicate corresponding parts in the figure.

### DETAILED DESCRIPTION

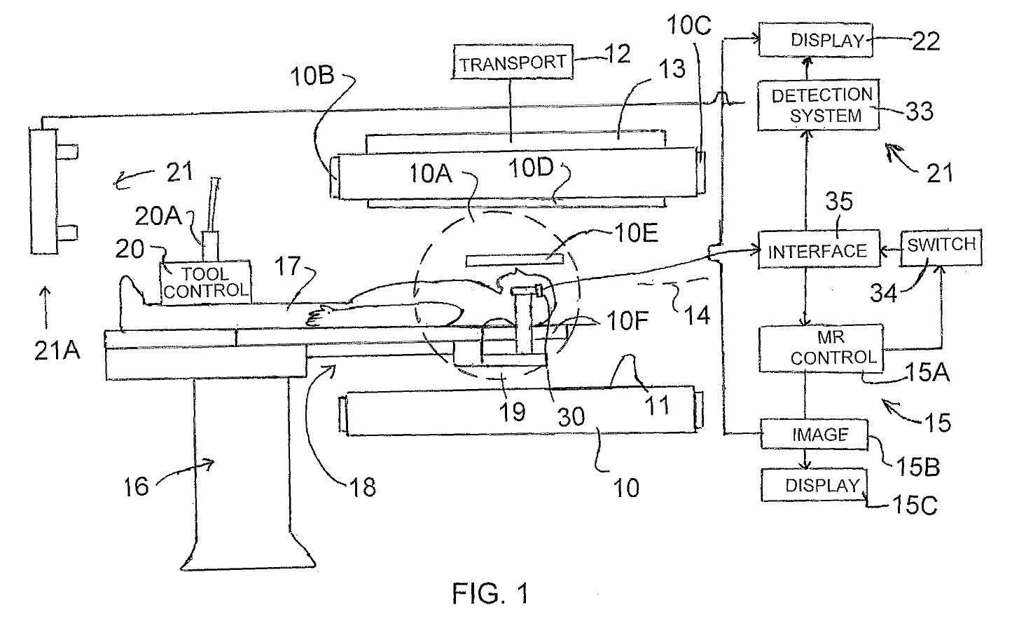

In FIG. 1 is shown an intra-operative magnetic resonance imagining system of the type shown in U.S. Pat. No. 5,735,278 (Hoult) filed Mar. 15, 1996, the disclosure of which is incorporated herein by reference.

This system includes a magnetic resonance imagining component which includes a magnet 10 of the type including a generally cylindrical body defining a horizontal bore 11 where the magnet is carried on a track 12 on a suitable mounting assembly 13 allowing the magnet to be moved longitudinally along its axis 14. The remainder of the imaging system is shown schematically at 15 and the details of suitable magnetic resonance imagining systems are of course well known to a person skilled in the art so that no description is necessary here.

A patient operating table is indicated at 16 on which a patient 17 can be placed for the various procedures. The table is arranged so that it can enter the bore as the front end 18 of the magnet is moved longitudinally along the axis 14 to engage over the table 16 with the patient 17 lying on the table.

Attached to the head of the patient is provided a head fixation device 19 again of a conventional nature. Such devices are well known to a person skilled in the art and attached to the head of the patient to hold the head stationary within the bore of the magnetic relative to the table so that the patient is maintained at a fixed position during the imagining procedure.

Commonly, the fixing fixation device 19 is of a nature which will allow the surgeon to carry out an operation using various tools as well known to a person skilled in the art. In the arrangement of the present invention, the tools are controlled by a tool control system generally indicated at 20 operated by an image guided surgery system generally indicated at 21.

Commonly the image guidance system 21 includes various controls to be operated by the surgeon together with a display which provides an image to the surgeon of the position of the tool relative to the anatomy of the patient.

In the present system the image of the anatomy of the patient is provided from the imaging system 15 obtained from the magnetic resonance imaging process.

It is necessary of course that the image from the imaging system 15 be registered relative to the position of the tool as controlled by the IGS system 21.

The magnetic resonance imagining system can be used repeatedly during the operation procedure so as to enable the surgeon to observe the effect of the surgery as it proceeds.

The MR imaging system includes the magnet 10 defining a primary magnetic field with an imaging space 10A within that magnetic field. Gradient coils 10B and 10C are provided as is well known for generating magnetic fields which vary during imaging.

RF signals are transmitted to the imaging space by antennae 10D and RF signals are received from the sample to be tested, in this case the body part of the patient within the imaging space by an RF receiving system generally including head coils 10E and 10F. In the example shown these include a top coil 10E and a bottom coil 10F with the former being removable during the operation procedure and the latter generally remaining in place.

The MR system is well known and includes many control systems and management systems but for convenience of illustration these are shown at 15A as the details are already well known. Thus schematically, the control system 15A operates the magnet and the RF system and includes software 15B for calculating from the signals received by the RF coil an image of the body part of the patient on a display 15C.

It will be appreciated that the control system 15A locates the image in co-ordinates of an image space which are calculated and relate to the space within the imaging zone where the data for the image is taken.

The image guidance system 21 is arranged for guiding the position of the tool 20A relative to the body part of the patient within a physical space defined by physical co-ordinates related to the physical location of the body part of the patient. The image guidance system is arranged to be used with the magnet retracted to a remote location where the magnetic field is beyond a position where it affects the body part. The image guidance system 21 includes a camera based detection system 21A for detecting the position of position markers.

The signals from the cameras 21A are received by and processed by a detection system 33 which transfers the detected positions to a display 22.

Thus the guidance control system acts to calculate a position of the marker on the tool relative to an image on the display and provides guidance information to a user of the position of the tool relative to the body part of the patient.

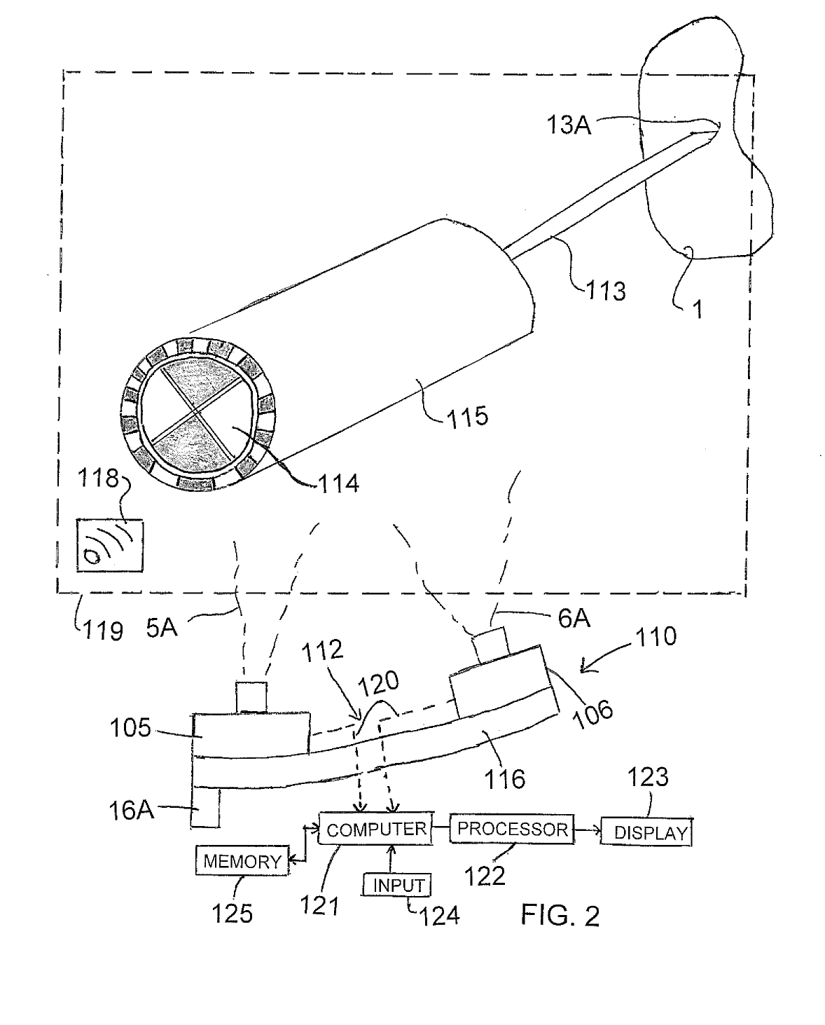

Referring to FIG. 2 the guidance control system 21 includes a tracking system 110 has an optical sensor assembly 112 used to track the relative position and orientation of a marker 114 attached to an object 115, such as but not limited to a surgical tool 113 tracked in relation to a portion of a patient's anatomy 1 in a surgical scene or field of view 119. The sensor assembly 112 is a stereo sensor having a first digital video camera sensor 105 with a first field of view 5A and a second digital video camera sensor 106 with a second partially overlapping field of view 6A. More than two cameras could also be used if desired. Suitable sensors or detector arrays for this purpose are commercially available. Such cameras are typically delivered pre-calibrated to allow the association of a pixel position in each of the images with a corresponding linear ray equation in a common sensor 3D space.

The position and orientation of the cameras with respect to one another are fixed by rigidly securing the cameras to a support frame 116, so as to provide for the overlapping views. The support frame 116 is securely mounted to a fixed support 116A, with an adjustable joint to provide adjustment capability to the direction and proximity of the field of views to the field of view 119 containing the patient anatomy 101 and the tool 113. The cameras have multiple and arbitrary line of sight vectors which are contained within their respective fields of view. The cameras may also have multiple fixed line of sight vectors, in which case the reference marker may not be required as the camera position is known and can therefore act as the reference. A source of illumination 118 can include light energy to supplement visible light already present at the scene, such as existing room lighting or sunlight.

The marker 114, as described in more detail hereinafter, is securely coupled to the tool 113, such that the projection images of marker 114 can be sensed by the cameras when positioned in the corresponding fields of view. The cameras record projections of all items in the scene 119. These image projections are oriented typically perpendicular to the lines of sight 131, 133. For example, a projected image representing the marker is recorded by the cameras 105 and 106 respectively. A series of image intensity signals 138 representing the projected images of the marker 114 are transmitted by the cameras 105, 106 to the computer 121, where the signals 120 are processed to calculate the three dimensional location of the center of each marker 114. These signals 120 contain image pixel information of the projected images for all the objects 115 and markers 114 present in the scene 119. It is noted that the projected images are typically located as formed on a sensor plane (not shown) inside the camera sensors 105, 106. Accordingly, the position and orientation of the tip 113A of the tool 113, relative to the position of the anatomy 1, is determined by a processor 122 of the computer 121 using the known spatial relationship between the marker 114 and the tip 13A, as the tool 113 is moved about the anatomy 1 in the surgical scene 19 during an operation, or about an anatomical model (not shown) for a simulated planning procedure. The position and orientation information is calculated using the image intensity values of the pixels contained in the signals 120. In this manner, the orientation of the specially marked tool 113 is tracked when moving unpredictably through the field of view 119. The orientation of the anatomy 1 and the tool 113 can be determined relative to a fixed reference point, such as the fixed support 16A.

The processor 122 is coupled to a display 123 and to user input devices 124, such as a keyboard, mouse, or other suitable devices. A computer readable storage medium 125 is coupled to the processor 122 for providing instructions to the processor 122 to perform steps or algorithms related to the determination of the relative spatial position of the tool 113 with respect to the anatomy 1, as well as monitoring the presentation of the anatomy 1 and tool 113 images on the display 123.

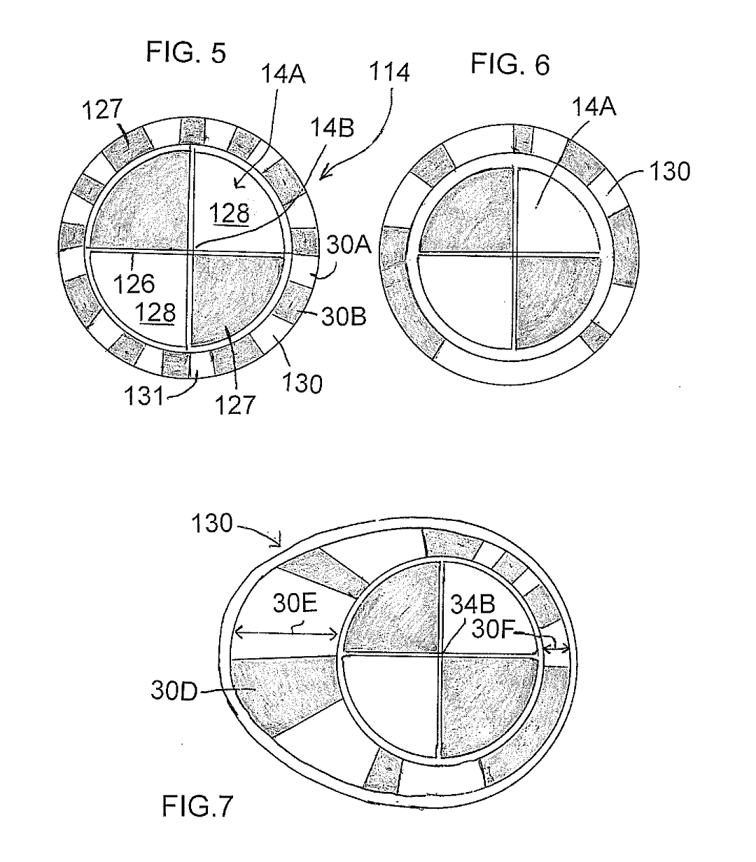

As shown in FIGS. 5, 6 and 7, further details of the markers 114 are provided. Each of the object markers 114 is made up of a target pattern 14A which is a visible high-contrast pattern appearing a surface of the marker 114. Each visible target pattern 14A has one feature point 14B which is an arrangement of light reflectance in the target pattern 14A which is arranged such that the target pattern 14A and the feature point 14B will be easy to detect using an orientation detection algorithm performed by the computer system 121 and the processor 122 under a wide range of rotation angles, sizes and lighting conditions of the marker 114.

The feature point 14B is defined as an intersection of straight edges 126 formed between alternating dark 127 and bright 128 regions. Such intersections do not commonly occur naturally in images and preferably maintain their pattern characteristics of being an intersection of straight edges 126 under all viewing angles, magnifications, blurring, and perspective distortion that can be encountered by the camera sensors in viewing the marker 114 in the scene 119.

These feature points 14B are also detectable from a wide range of possible appearances in the image using an orientation detection algorithm. For example, stepping along a circle surrounding the feature point 14B will yield a fixed number of alternating intensity peaks and valleys and the direction of the intensity gradients at the strongest edges between each such peak and valley pair is substantially tangential to the circle. The peaks and valleys of the intensity gradients result from the alternation of dark 127 and light 128 contrast regions located within the circle.

The pattern thus includes first components 126 and 127 arranged such that the processor is able to detect a specific location on the marker, that is, the feature point 14B which defines a center of the marker. In the present arrangement, the marker can operate with only a single feature point 14B.

The pattern further includes second components 30 which are arranged to be unique to the marker 14 concerned and thus are different from other markers of the system. These second components are arranged such that the processor is able to distinguish the marker concerned from the other markers. As shown in FIGS. 5, 6 and 7, the second components are arranged in an array 131 extending partly or wholly around the feature point 14B. In the embodiments shown, the array 131 extends fully around the feature point 14B as a complete ring centered on the feature point 14B. The second components 130 define a series of bars of light and dark 30A and 30B of different width centered around the location or feature point 14B. These thus form different sized curved line segments centered around the location which allows an encoding based on the length and number of the line segments, such as a typical 2D bar code.

The fact that the components to be scanned and analyzed are arranged in an array or ring centered on the feature point 14B allows the analysis system defined by the processor to locate the feature point 14B as a first step and then to look for the components around the center defined by the found feature point 14B.

As shown in FIG. 7, the pattern 30 also can include components of the array arranged around the feature point 14B arranged to define a base line 30D such that the processor is able to detect an angle of rotation of the pattern around the location. Thus the analysis system can locate on the pattern 130 the base line 30D and can measure the angle of rotation of this base line relative to a nominal axis in the detection array

In FIG. 7, the pattern 130 is offset to one side of the location so as to define a larger dimension 30E on the side defining an axis for determining the rotation around the location as opposed to the opposite side 30F. Thus the system looks for the array around the point 14B and analyzes it for the encoded data defining the unique marker and then also for the location and orientation of the base line 30D.

In this way, the marker is arranged such that the detector array and the processor can identify and measure the translational and rotational position of the marker.

As shown in FIG. 6, there is shown an alternative encoding system which uses colour information in the pattern 14A and/or in the pattern 130 to uniquely identify the marker.

The camera technology used in the cameras can be monochromatic, full colour or can use only one of the colour channels.

The object can be a surgical tool as shown. The object can also be non-surgical equipment such as booms, MRI, C-Arms and lights. The object can also be a component of a surgical robotic system, where knowing the precise location of the end effector is critical and where end effector real estate is limited.

The markers are preferably flat disks as shown with the patterns located in a common flat plane of the marker face. However the markers can also have a curved or spherical face.

In a further arrangement (not shown) there is provided a further embodiments of marker as used in the system of FIG. 1 where a part of the front face of the marker is raised or recessed so that three dimensional information can be used by detecting the raised and/or recessed sections to uniquely identify the marker and/or to detect the angular position of the marker. Thus the three dimensional information is provided by raised areas and/or depressed areas of relief on the marker.

In another arrangement (not shown) there is provided a series of markers 14 which can be selected by a user touching or pointing to a selected one of the markers. The processor is arranged to use information identifying each of a series of the markers as an input for process control. This can be used for a virtual keyboard or for gesture control.

Thus the processor is arranged to detect which marker is being selected by determining an area of occlusion of the marker in the image, such as for example by the location of the user's finger over the marker so that the camera system can no longer see the whole of the marker in the image. It will be appreciated that the arrangement and identification of the markers is carried out by the system prior to the selection by the user. On prompting by the system or as required, the user then occludes a part of one of the markers as shown which is detected by the processor and used as an input such as a switch or a keystroke in accordance with the programming of the system.

Also, the occlusion of the marker can be used to input a graded or variable input, such as a volume control, by the user acting to move the area of occlusion on the marker. For example the user may rotate the finger around the center of the marker to input a degree of increase or decrease of a value indicated by the marker. The processor is arranged to detect the movement and to use the movement as an input for process control.

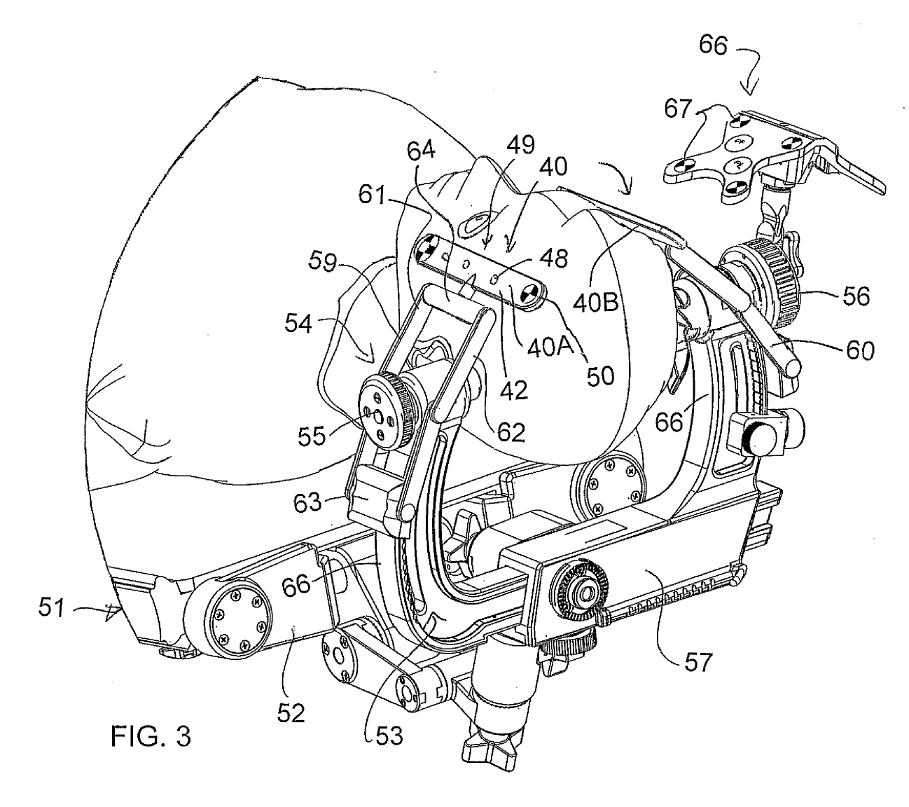

In order to provide the registration between the image of the imaging system 15 and the position of the anatomy of the patient, there is provided a common fixture 40 shown in FIG. 3.

The common fixture 40 comprises two separate plates 40A and 40B. The plates are formed from a plastics material which is invisible in MR imaging. The plates carry a plurality of MR markers 48 arranged in an array 49 with the markers spaced across the plate. The markers can be formed simply by drilling a hole in the plate and filling the hole with a suitable material which is visible in MR imaging. The size of the drilled holes and therefore the plugs which form the MR visible material can vary but are typically sufficiently small and spaced so as to avoid interfering with the structural strength of the plate. The array is arranged so that the position of the array can be determined when only some of the MR markers are visible in the MR image.

In addition the MR markers in the array are arranged so that the markers of one plate are different from the markers of another plate so that the image can be analyzed to determine from the image which one of a series of such plates is in use in the imaging process.

Thus in use the fixture is located on the patient table and is used in the MR imaging so that the array 49 of markers 48 appears in the image obtained in the MR imaging co-ordinates system. Thus the image of the patient obtained in the MR imaging system also includes the image of the array 49. Depending upon the part of the patient which is being imaged some or all of the array 49 may appear in the image. From an irregular pattern defined by the array, an analysis of the image obtained can determine the location of the whole array and also can determine the particular fixture 40 in use.

For example the array may have the following characteristics to enable the above analysis to be determined. The set of distances between a pair of markers in the array contains no duplicates; this allows the algorithm to uniquely identify all markers as long as at least three are detected in the image.

The common fixture 40 further includes a marker 50 which is located on a face 42 of the common fixture 40 so that it is visible by the above camera detection system 110. The fixture 40 may carry one or more of the markers 50. If the marker is of the type described above, which provides both encoding of the particular marker in use and also the angular orientation defined by the axis 30D of the marker, then a single marker can be used.

In the alternative a plurality of visual markers 50 can be provided along the front face or at other locations on the fixture 40 so that the imaging system 110 can determine the position of the fixture 40 in the imaging co-ordinates. In both cases, the markers act to identify in the image obtained by the processor 122 the location of the fixture 40.

As the position of the marker 50 relative to the array 49 is predetermined and fixed, an analysis can be carried out by the detection system 33 of FIG. 1 to determine the position of the fixture 40 in both the MR imaging co-ordinates and the tracking imaging co-ordinates to provide an automatic registration of the images from the MR imaging system relative to the images from the tracking system.

The software necessary to carry out such an analysis and registration is relatively straight forward and is certainly within the skill of a person skilled in this general art.

Turning now to FIG. 3, the system includes a patient table 51 which is arranged both for MR imaging and for surgical procedures. A table of the type described and disclosed in application Ser. No. 12/333,032 filed Dec. 11, 2008 and published as Publication No. 2009/0306495 on Dec. 10, 2009, by the present assignees, can be suitable and the disclosure of this application is incorporated herein by reference.

The table 51 includes a head end 52 to which is attached a head fixation device 53 of a conventional nature. The head fixation device 53 includes a clamp system 54 with a clamp element 55 on one side and a second clamp element 56 on the other side with these two sides connected by an adjustable clamp element 57 to pull the clamp elements 55 and 56 together to clamp the skull of the patient therebetween. Arrangements of this type are well known and provide pins (not shown) which engage into the skull of the patient to hold the skull in a fixed position during imaging and during the surgical procedures.

In this case the head fixation device is modified to include a bracket 59 on one side and a bracket 60 on the other side which provide supports for the plate 40A and 40B of the common fixture 40 which are mounted on the head fixture device by those brackets. The brackets 59 and 60 are located around the clamp elements 55 and 56. The brackets 59 and 60 engage the plates 40A and 40B so as to hold those in a fixed position relative to the head fixation device 54. Each bracket 59, 60 includes a pair of arms 61, 62 mounted on either side of a support block 63. A bridging member 64 connects the arms at their outer end and attaches to a respective one of the plates 59, 60. The block 63 is carried on clamp arms 66 of the head fixation device 53 and are connected by a suitable mounting which allow the blocks to be removed readily from the clamp.

The brackets 59 and 60 are arranged so that the plate 40A and 40B of the common fixture 40 can be inserted into place with the head of the patient already attached to the head fixation device. In this way the common fixture 40 can be inserted into place once the patient is located on the table and the head of the patient is fixed in position in the head fixation device on the table.

Thus the markers are carried on at least one plate carried on a mounting device which is moved to provide adjustment of the position of the plate relative to patient while holding the plate stationary during the imaging. This is achieved by moving the arms 61, 62 which hinge at the middle and at the mounting block allowing the plates to be moved in and out away from the head of the patient. The hinges are sufficiently stiff to hold the arms in place after adjustment so that the plates do not sit on the patient. The arms 61 and 62 are located to span either side of the clamp 55 so that the plates and their mounting can be connected independently of the operation and adjustment of the head clamp. The plate and the mounting block 63 are removed after imaging.

Once located in place adjacent the head of the patient, the common fixture remains fixed in place during imaging. The blocks and arms 61 and 62 hold the plates so that each remains in a fixed position on a respective side relative to the head during the imaging. The plates and their mounting can be removed for the surgical procedure and replaced when required for imaging carried out intra operatively. Each time the plates are replaced, the MR markers 48 can be located in the image and the visible markers 50 can be located in the imaging system so as to provide the required registration.

The plates are located each on a respective side of the head fixation device so that they do not interfere with the head or the anterior or posterior RF coils. An open area behind or underneath the head is sufficient to receive the posterior coil of the RF coil system of the MR imaging system as shown in the end elevational view of FIG. 3.

In an alternative arrangement (not shown) the mounting device for the plates 40A and 40B or the plates themselves can be mounted on a robotic arm which allows the plates to be moved by the movement of the arm under control of the operating system from a deployed position to a retracted position. In this way the plates are supported in place during the imaging and registration steps but then can be retracted automatically when the surgical procedure is under way with the image guidance relying on the registrations provided by the stationary marker which remains in place at the head clamp.

The posterior RF coil can remain in place during the surgical procedure. The coil may be removed in some circumstances.

The provision of the marker 50 allows this to be visible to the imaging system even after draping of the patient with the necessary surgical drapes for the surgical procedure. The drapes therefore are applied over the head of the patient but the plates can be located over the drapes when attached. The drapes thus can remain properly in place during the surgical procedure without interfering with the imaging of the plates in the MR system and in the image guidance system.

In addition a fixed reference 66 is provided on the head fixation device and stands upwardly therefrom at a position fixed relative to the head fixation device so that this reference location is readily visible at all times in the tracking imaging system. The tracking of the tool 115 in the surgical procedure is thus carried out using one or more markers 67 on the fixed reference 66 rather than the marker 50 on the common fixture 40.

In an alternative arrangement (not shown) the markers 48 and 50 can be located on a curved sheet located underneath the head of the patient and carried on a support bridging the clamp arms 66 of the head fixation device 53. This sheet typically would remain in place during imaging and during the surgical procedure since it is located in the same position as the posterior coil and thus does not interfere with the procedure.

The sheet can include an aperture extending through the sheet from the upper surface to the lower surface at a position spaced from the array 49 of markers 48. The aperture is located at a position which is suitable for allowing access to the head of the patient during the surgical procedure, if necessary. The aperture can be rectangular but it will be appreciated that any such aperture shape can be used as required. Different sheets may be provided with different apertures for different purposes and again these are identified by the unique marker 50 and by the unique array 49.

While the common fixture 40 as shown is designed particularly for attachment to the head fixation device for use in surgical procedures on the head of the patient, it will be appreciated that other such common fixtures 40 can be designed for use with other parts of the body of the patient and can be located at other positions relative to the table 51. In all cases there is provided an arrangement for locating the patient in fixed position and for locating the common fixture 40 in relation to that fixed position defined by the locating system.

In all cases the common fixture is located and shaped so that it can remain in place during the imaging procedure so as to provide the common location between the array 49 and the marker or markers 50 so as to provide automatic registration of the images so as to allow guidance of the tool 115 relative to the MR image for proper tracking of the tool to provide the most accurate procedures on the imaged body part of the patient.

A further alternative arrangement of the common fixture (not shown) can be arranged for clamping onto the posterior coil. In this arrangement the common fixture or common registration element has the common registration element markers located on the side edges so as to face upwards on either side of the patient. The common fixture comprises a concave sheet curved around an axis longitudinal of the patient which is formed of a plastics material which is not visible in the image with a pattern of drilled holes containing an image visible material. The sheet has clamping brackets and by which it is attached to the posterior coil and is arranged to fit underneath the fixture and to be removable while the posterior coil remains in place. The bracket is arranged to butt one end of the coil and pivotal toggle members forming a bracket are pivotal into locking positions to clamp onto the end of the posterior coil. The coil is carried on a head clamp 53 of a conventional construction by clamping onto a cross arm 57 of the clamp. Thus this common registration element is arranged such that it can be moved into place after the patient is pinned into the head fixation device and can remain for the duration of the operation.

The markers are located on the longitudinal side edges of the sheet so as to face upwardly to be exposed to a camera system above the patient. There is also provided a separate fixed reference carried separately on the head clamp having a visible positioning marker, the location of which can be determined in the image guidance system. The separate fixed reference is used for the image guidance system rather than the markers after the location of the reference relative to the markers has been determined by the image guidance system. In this way the patient can be draped by drapes which cover the head and the posterior coil and the head clamp and also the side edges carrying the markers. While all of these elements are covered, the separate fixed reference remains exposed when the patient is draped for surgery.

In order to ensure that no movement has occurred with would interfere with the registration of the images, there can be provided a plurality of divots or recesses in the sheet that can be located by physical contact through the drapes. In this way a quick check can be carried out that the common registration device and the reference are still in registration by pointing to the divots using the surgical tool. In this way the system can be operated to detect and correct for movement of one of the fiducial frames or fiducial frame segments using the tracking system markers on each fiducial frame or segment. The system can use the co-ordinate reference frame or can add tracking system markers on the skull clamp to achieve the same purpose.

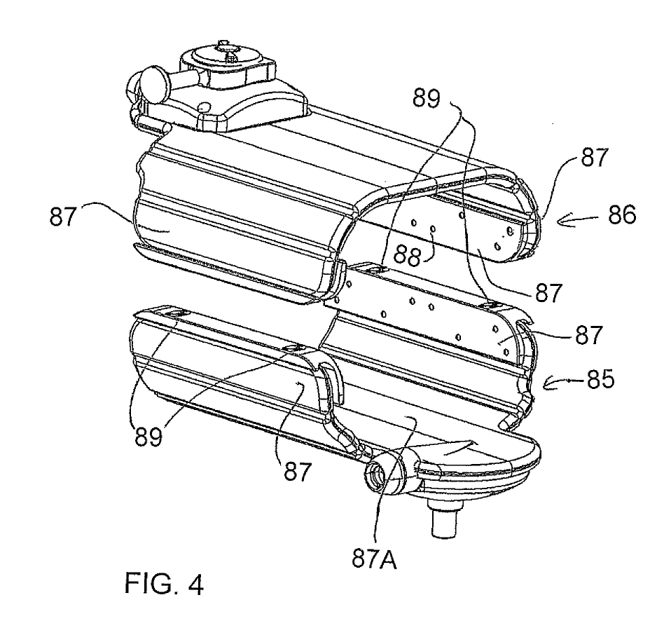

In FIG. 4 is shown a posterior RF coil which is flexible as indicated at 85. There is also provided an anterior coil 86 which is also flexible. Such flexible coils are well known and are becoming more important in the industry. The arrangement herein thus provides multiple fiducial frames or common fixtures 87 one on the top coil and one on the bottom coil. In order to accommodate the flexing of the coils to match a required curvature for the patient, the common fixture includes multiple fiducial frame segments 87 on each single, flexible coil. Each fiducial frame segment includes a number of both MR markers 88 and at least one tracking system marker 89 of which two markers 89 are shown on each segment 87. An additional segment may also be provided in the center as indicated at 87A since this adds more of the markers at positions spaced around the head of the patient to provide more accuracy and some redundancy in the marker positions.

In the embodiment of FIG. 4, the flexible coils 85 and 86 flex only in a transverse direction around a longitudinal axis and are stiff in the longitudinal direction. Thus the segments are strips extending along the full length of the coil. If the coil is flexible in both directions the segments or flat plates can be formed in array of “tiles” in rows and columns across the coil surface allowing the required flexing by movement of the tiles relative to one another while the tiles remain flat. Each tile is then tracked in MR and tracking image to locate the positions of the coil. Thus the system is arranged to detect and correct for movement of one of the fiducial frames or individual tiles using the tracking system markers on each fiducial frame.

In the limit, there could be only a single marker that is visible to both the tracking system and to the MR; so that the markers are attached to the flexible coil.

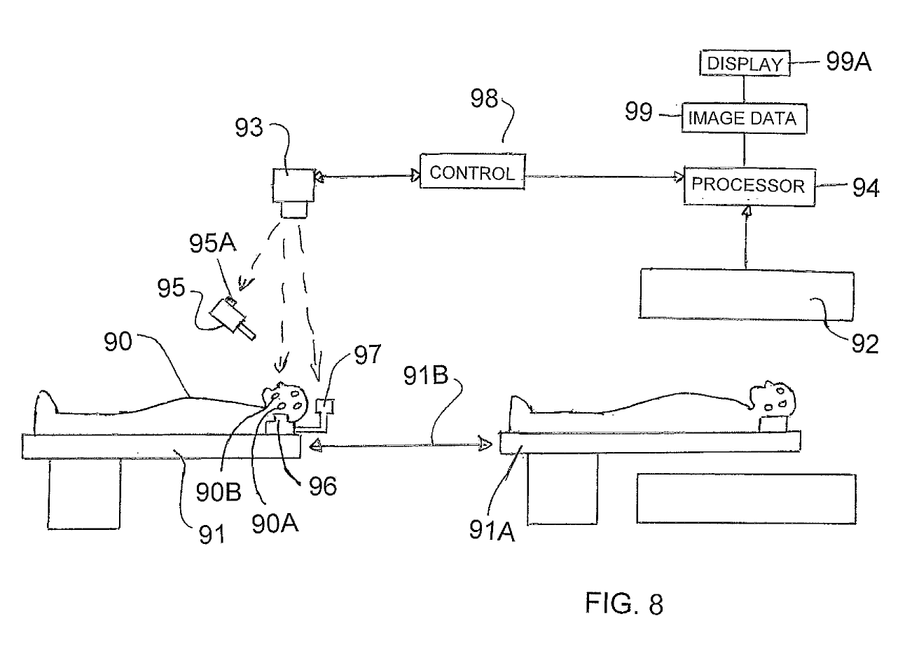

Turning now to FIG. 8, a further alternative system is shown where the patient 90 is supported on a table 91 during surgery and on a table 91A during imaging. The patient then travels between the locations as shown at 91B for the separate imaging and surgery in different locations and possibly at different times.

The MR imaging system is shown schematically by the magnet 92 and by a processor system which operates the imaging as indicated at 94.

The tracking system is shown schematically by the camera 93 and the tool 95 which carries a marker or markers 95A. The head clamp 96 carries a reference marker 97 as previously described.

A common control system 98 controls the tracking system and the imaging system and is arranged to generate image data 99 for display at 99A.

In this arrangement, pre-operative MR images at the separate location are obtained. Prior to the imaging, optical markers 90A visible in the camera system 93 of the image guidance system are located on or adjacent MR visible markers 90B that are placed in the MR field of view on the patient before the pre-operative MR images are acquired. The optical markers at or adjacent the MR markers and the known relative positions therebetween allow the automatic identification of the MR markers in the optical co-ordinate space. The optical markers can be of the type described in detail above in relation to FIGS. 8 to 10. Thus the marker can comprise a generally flat disk with an adhesive backing which attaches it to the skin and with the optically visible arrangement described above printed on or attached to the front surface. In a center of the disk is provided a component of MR visible material. Preferably this is raised slightly from the skin of the patient by a distance of 1-2 mm so as to allow the algorithm to more easily and more accurately calculate the necessary positions and transforms.

For both intra-operative and pre-operative MR images, a computer algorithm is used as part of the automatic registration system to automatically identify the optical and MR markers, and calculate the co-ordinate mapping between the two. This allows the automatic registration and therefore enables navigation without manual registration.

Thus the system uses a number of attachable fiducial markers arranged to be attached to the patient that can each be detected in the imaging system and by the tracking system and the system uses marker recognition to perform automatic registration.

Between the optical and MR markers there is provided a spacer (not shown) not visible in the imaging to ensure that the image marker is located at a position spaced away from the skin during imaging. The algorithm is arranged for use with an arbitrary arrangement of such attachable markers so that it can accommodate random or individually selected positions of the markers without requiring specific attachment points.

The head is not pinned during the preliminary imaging at location 91A, but the attachable fiducial markers are kept in place between the time of the scan and the time at which the patient is on the table 91 and pinned. The attachable markers can be attached using adhesive, screw into bone, or the like.

In order to efficiently and accurately use the MR imaging system, the control system 98 can be arranged to carry our separate marker-sensing scans and an anatomical scan, which the navigation software provides. For example, the system can use one MR pulse sequence to image the relevant anatomy at high resolution and a different MR pulse sequence to image the space outside the head where the markers are located. The system may even utilize different MR coils for the scans. The navigation system 98 can be used to drive the scanner acquisition in this mode or the scans may be effected under the control 94 of the MR imaging system.

Since various modifications can be made in my invention as herein above described, and many apparently widely different embodiments of same made within the spirit and scope of the claims without department from such spirit and scope, it is intended that all matter contained in the accompanying specification shall be interpreted as illustrative only and not in a limiting sense.

1. A method for registering images, comprising:
obtaining at least one image of a part of the body of the patient in an imaging system;
the imaging system defining an image co-ordinate system;
placing image visible positioning markers at positions on or adjacent the part of the patient so as to be visible in the image;
obtaining a series of location images of one or more elements adjacent part of the patient in an image guidance system for providing location information relating to the elements;
the image guidance system defining a location co-ordinate system;
placing at least one guidance system positioning marker visible in the location image at respective positions on or adjacent the part of the patient so as to be visible in the location image, the image visible positioning markers being located at predetermined known locations relative to said at least one guidance system positioning marker in the image guidance system;
and providing automatic registration between the image co-ordinate system and the location image co-ordinate system by using data relating to the relative positions of the image visible positioning markers and said at least one guidance system positioning marker.

2. The method according to claim 1 wherein the automatic registration is carried out by a control system from the images, without manual intervention by the user, for each image set acquired

3. The method according to claim 1 wherein the image visible positioning markers and said at least one guidance system positioning marker are separate markers independent of one another and are placed on a common registration element.

4. The method according to claim 3 wherein the common registration element is located underneath the part of the patient such that the element can remain in place during the imaging and the location imaging.

5. The method according to claim 3 wherein the registration element markers are located on the common registration element so as to face upwards on either side of the patient.

6. The method according to claim 3 wherein the common registration element is formed of a plastics material which is not visible in the image with a pattern of drilled holes containing an image visible material.

7. The method according to claim 3 wherein the common registration element is attached to a head fixation device.

8. The method according to claim 3 wherein the common registration element is arranged such that it can be moved into place after the patient is pinned into the head fixation device.

9. The method according to claim 3 wherein the imaging system is an MR system including a posterior RF coil and wherein the common registration element is separate from the posterior coil.

10. The method according to claim 3 wherein the imaging system is an MR system including a posterior RF coil and wherein the common registration element is arranged to clamp onto the posterior coil.

11. The method according to claim 1 wherein the image guidance system comprises a visible optical system using cameras for detecting the positioning markers using visible light.

12. The method according to claim 1 wherein the image visible positioning markers are arranged in a non-regular array.

13. The method according to claim 12 wherein the non-regular array is arranged to allow location of the array relative to the common registration element when only part of the common fixture is visible in the MR image.

14. The method according to claim 12 wherein the non-regular array is different for different ones of a plurality of common registration elements and a determination is made as to the different fixtures by analyzing the array.

15. The method according to claim 1 wherein there is provided a separate fixed reference having a visible positioning marker, the location of which can be determined in the image guidance system, wherein the separate fixed reference is used for the image guidance system rather than the markers after the location of the reference relative to the markers has been determined by the image guidance system and wherein the separate fixed reference remains exposed when the patient is draped for surgery.

16. The method according to claim 1 wherein the MR imaging is carried out intra-operatively to obtain a new MR image and registration of the new MR image with the guidance image is carried out automatically, that is without manual intervention, for each MR image set acquired.

17. The method according to claim 1 wherein the imaging system is an MR system including an RF coil, wherein the RF coil is arranged to flex and wherein the common registration element includes a plurality of flat panels attached to the flex coil.

18. The method according to claim 1 wherein the image visible positioning markers are located in an image of the part of the patient by carrying out at least one separate marker-sensing scan used for locating only, which scan is separate from one or more anatomical scans which are used to generate an anatomical image of the part of the patient.

19. The method according to claim 18 wherein the image is obtained by MR imaging and wherein the separate marker sensing scan is carried out using separate MR coils for the scan.

20. The method according to claim 1 wherein the system uses a number of attachable fiducial markers arranged to be attached to the patient that can each be detected in the imaging system and by the tracking system.

21. The method according to claim 20 wherein there is provided a spacer not visible in imaging to ensure that the image marker is located away from the skin during imaging.

22. The method according to claim 21 wherein the part of the patient remains unfixed during imaging, but the attachable fiducial markers are kept in place between the time of the scan and the time at which the part patient is pinned.

23. The method according to claim 21 wherein the attachable markers are attached to the body of the patient using adhesive or screw into bone.

24. The method according to claim 1 wherein the markers are carried on at least one plate carried on a mounting device which is moved to provide adjustment of the position of the plate relative to patient while holding the plate stationary during the imaging.

25. The method according to claim 24 wherein the plate and the mounting device are removed after imaging.

26. The method according to claim 24 wherein the at least one plate comprises a pair of plates each mounted on a respective side of the patient.

27. The method according to claim 24 wherein the mounting device is removably mounted on a head clamp.

28. The method according to claim 24 wherein the mounting device is mounted on a robotic arm.

29. A method for registering images, comprising:
obtaining at least one image of a part of the body of the patient in an imaging system;
the imaging system defining an image co-ordinate system;
placing image visible positioning markers at positions on or adjacent the part of the patient so as to be visible in the image;
obtaining a series of location images of one or more elements adjacent part of the patient in an image guidance system for providing location information relating to the elements;
the image guidance system defining a location co-ordinate system;
placing at least one guidance system positioning marker visible in the location image at respective positions adjacent the part of the patient so as to be visible in the location image, the image visible positioning markers being located at predetermined known locations relative to said at least one guidance system positioning marker in the image guidance system;
and providing registration between the image co-ordinate system and the location image co-ordinate system by using data relating to the relative positions of the image visible positioning markers and said at least one guidance system positioning marker;
wherein the imaging is carried out intra-operatively to obtain a new image and registration of the new image with the guidance image is carried out automatically, that is without manual intervention, for each image set acquired

30. A method for registering images, comprising:
obtaining at least one image of a part of the body of the patient in an imaging system;
the imaging system defining an image co-ordinate system;
placing image visible positioning markers at positions adjacent the part of the patient so as to be visible in the image;
obtaining a series of location images of one or more elements adjacent part of the patient in an image guidance system for providing location information relating to the elements;
the image guidance system defining a location co-ordinate system;
placing at least one guidance system positioning marker visible in the location image at respective positions adjacent the part of the patient so as to be visible in the location image, the image visible positioning markers being located at predetermined known locations relative to said at least one guidance system positioning marker in the image guidance system;
and providing registration between the image co-ordinate system and the location image co-ordinate system by using data relating to the relative positions of the image visible positioning markers and said at least one guidance system positioning marker;
wherein the image visible positioning markers are located in an image of the part of the patient by carrying out at least one separate marker-sensing scan used for locating only which scan is separate from one or more anatomical scans which are used to generate an anatomical image of the part of the patient.
